SEQUENCE LISTING (16 sequences)

SEQ ID NO: 1 - protein, 418 aa, Homo sapiens [sequence omitted]

SEQ ID NO: 2 - protein, 14 aa, Homo sapiens
LAAAVSNFGYDLYR

SEQ ID NO: 3 - protein, 7 aa, Homo sapiens
TESIIHR

SEQ ID NO: 4 - protein, 17 aa, Homo sapiens
ALYYDLISSPDIHGTYK

SEQ ID NO: 5 - protein, 11 aa, Homo sapiens
ELLDTVTAPQK

SEQ ID NO: 6 - protein, 5 aa, Homo sapiens
IVFEK

SEQ ID NO: 7 - protein, 9 aa, Homo sapiens
SSFVAPLEK

SEQ ID NO: 8 - protein, 7 aa, Homo sapiens
VLTGNPR

SEQ ID NO: 9 - protein, 15 aa, Homo sapiens
LDLQEINNWVQACMK

SEQ ID NO: 10 - protein, 13 aa, Homo sapiens
KTSLEDFYLDEER

SEQ ID NO: 11 - protein, 12 aa, Homo sapiens
TSLEDFYLDEER

SEQ ID NO: 12 - protein, 8 aa, Homo sapiens
VPMMSDPK

SEQ ID NO: 13 - protein, 9 aa, Homo sapiens
LSYEGEVTK

SEQ ID NO: 14 - protein, 12 aa, Homo sapiens
LQSLFDSPDFSK

SEQ ID NO: 15 - protein, 40 aa, Homo sapiens
AGFEWNEDGAGTTPSPGLQPAHLTFPLDYHLNQPFIFVLR

SEQ ID NO: 16 - protein, 12 aa, Homo sapiens
DTDTGALLFIGK

(19) United States
(12) Patent Application Publication
(10) Pub. No.: US 2005/0148508 A1
(43) Pub. Date: Jul. 7, 2005

(54) Pigment epithelium derived factor from human plasma and methods of use thereof

(76) Inventors: Shmuel Shaltiel, Rehovot (IL); Iris Schvartz, Yavne (IL)

Correspondence Address: WINSTON & STRAWN LLP, 1700 K STREET, N.W., WASHINGTON, DC, 20006, US

(21) Appl. No.: 10/882,638

(22) Filed: Jul. 2, 2004

(30) Foreign Application Priority Data
Jan. 3, 2002 (IL) 147444

(51) Int. Cl. G01N 33/53; C07K 14/475; A61K 38/18; C07H 21/04

(52) U.S. Cl. 514/12; 530/399; 435/69.1; 435/320.1; 435/325; 536/23.2; 435/7.1

(57) ABSTRACT

Polypeptides of pigment epithelium derived factor (PEDF) isolated from human plasma and fragments thereof, methods for preparing them, pharmaceutical compositions containing them and methods for diagnosis and treatment of angiogenesis-related diseases using such polypeptides.

### CROSS-REFERENCE TO RELATED APPLICATIONS

This application is a continuation of International application PCT/IL03/00007 filed Jan. 2, 2003, the entire content of which is expressly incorporated herein by reference thereto.

### FIELD OF THE INVENTION

The present invention relates to pigment epithelium derived factor (PEDF) isolated from human plasma and to fragments thereof, to pharmaceutical compositions thereof, and to methods for diagnosis and treatment of angiogenesis-related diseases using such polypeptides.

### BACKGROUND OF THE INVENTION

Pigment epithelium derived factor (PEDF) was originally identified in the culture medium of pigment epithelial cells obtained from fetal human retina. Based on its DNA and amino acid sequence it was concluded that it belongs to the superfamily of the serine protease inhibitors (serpins) (Steele et al., 1993). Analysis of the folded conformation of serpins indicated that they all contain an exposed peptide loop at their C-terminal edge that is highly susceptible to proteolysis. Upon cleavage by the appropriate proteinase the inhibitory serpins undergo a conformational change, which is characterized by a transition from a stressed (S) into a relaxed (R) loop structure. Although PEDF shares a ˜30% homology with the serpins, it behaves like a non-inhibitory member of the serpin superfamily, as it does not undergo the S to R conformational change.

PEDF was first described as a neurotrophic factor with an apparent molecular weight of 50 kDa that induces a specific neuronal phenotype in retinoblastoma cells. This morphological change was shown to be accompanied by an increased expression of the neuron-specific enolase, and of the 200 kDa neurofilament subunit (Steele et al., 1993). The neurotrophic activity of PEDF was also demonstrated by its ability to support neuron survival (e.g., cerebellar granule neurons, and hippocampal neurons. It was also shown to protect neurons against neurotoxic effects. Structure-function studies have shown that this neurotrophic activity is located at the amino terminus (amino acid residues 78-121) of PEDF, and that it is mediated through a ˜80 kDa membranal receptor, which is abundant in retinoblastoma cells.

The cDNA of PEDF was found to be essentially identical with the early population doubling cDNA-1 (EPC-1) in fibroblasts (Pignolo et al., 1993). The mRNA expression of the EPC-1 cDNA was shown to be induced under conditions of growth arrest, due to either serum deprivation or to a density-dependent contact inhibition in early passages of WI-38 fibroblasts, but not in senescent WI-38 fibroblasts. In addition, PEDF was shown to inhibit the proliferation of endometrial carcinoma ephithelial cells (Palmieri et al., 1999). These findings raise the possibility that PEDF may play a blocking regulatory role in cancer cell growth and that the age dependent drop of EPC-1/PEDF expression and secretion may be involved in the well known senescence-related increase of cancer incidence.

One of the most intriguing properties of PEDF is its activity as a very potent inhibitor of angiogenesis. It inhibits not only the bFGF-induced migration of endothelial cells in vitro, but also the bFGF-induced neovascularization in an avascular rat cornea. Furthermore, addition of anti-PEDF antibodies to rat corneas was shown to stimulate the invasion of new vessels into these comeas, suggesting that PEDF plays a physiological regulatory role in retinal angiogenesis (Dawson et al., 1999). Indeed, systemic administration of PEDF was recently shown to prevent retinal neovascularization in a murine model of ischemia-induced retinopathy (Stellmach et al., 2001). PEDF appears to inhibit retinal angiogenesis by causing apoptosis of endothelial cells in vitro and in vivo (Stellmach et al., 2001). The inhibition of retinal neovascularization was further confirmed by the finding that intravitreous injection of PEDF reduced choroidal and retinal neovascularization (Mori et al., 2001).

Angiogenesis (neovascularization) plays a crucial role in physiological processes including embryogenesis and tissue repair, but is also involved in pathological processes, including diabetic retinopathy, age-related macular degeneration, and tumor growth. In diabetic retinopathy, new blood vessels grow into the retina, and cause blindness. In age-related macular degeneration, a leading cause of visual loss in the elderly, new blood vessels grow through the Bruch's membrane and invade the retina, thus impairing vision. In tumor angiogenesis, new blood vessels grow from surrounding tissue and supply the tumor with required nutrients and oxygen, thus supporting an increase in the tumor mass.

The PEDF used in the studies described above was obtained either from the interphotoreceptor matrix of hundreds of bovine eyes or from recombinant sources using bacterial or mammalian expression systems in which human PEDF was produced. For example, Stratikos et al. described the expression of human recombinant PEDF carrying a histidine tag in CHO cells (Stratikos et al., 1996). This recombinant human PEDF was found to be functionally identical to bovine PEDF in the differentiation of retinoblastoma cells and in the blocking of neovascularization.

U.S. Pat. No. 5,840,686 to Chader et al., discloses nucleic acids that encode for PEDF and for a truncated PEDF, the equivalent proteins and methods for producing recombinant PEDF and the truncated PEDF. U.S. Pat. No. 5,840,686 claims a method of prolonging neuronal cell survival and a method for inhibiting glial cell proliferation using recombinant PEDF. U.S. Pat. No. 6,319,687 to Chader et al., claims a recombinant PEDF protein (418 amino acids) and truncated forms of PEDF having neurotrophic as well as gliastatic activity.

PCT Application WO 01/58494 claims a method of treating an ocular-related disease in an animal. The method comprises expression of an angiogenesis inhibitor and a neurotrophic agent in an ocular cell using an expression vector that contains the nucleotide sequence for these factors. A preferred angiogenesis inhibitor is PEDF, which is known to exert both anti-angiogenic and neurotrophic activities.

U.S. Pat. No. 6,451,763 to Tombran-Tink et al., describes the purification of PEDF from culture medium of human retinal pigment epithelial cells and claims methods of treating retinal diseases such as retinal tumors, macular degeneration, and diabetic retinopathy. It is also disclosed that in addition to retinal pigment epithelial cells, PEDF may be isolated from the vitreous humor of human, bovine, monkey and other primates. Since PEDF is abundant in the vitreous and since the vitreous is easily removed from the eyecup, the vitreous is suggested to be the easiest source from which PEDF can be isolated.

Nowhere in the background art is it disclosed that native human PEDF can be obtained from a readily available and abundant source such as human plasma. It is important to note that blood is a convenient source for obtaining biological materials since it is already in wide clinical use, for example in blood transfusion and as a source for several proteins and factors.

### SUMMARY OF THE INVENTION

It is now disclosed in accordance with the present invention that PEDF is present in the plasma of various animals and can be obtained therefrom in a homogeneous form. The present invention thus relates, in one embodiment, to isolated PEDF obtained from plasma. Preferably, isolated PEDF is obtained from plasma of a mammalian source.

In a more preferred embodiment of the invention, isolated PEDF is obtained from human plasma. According to the present invention, the isolated human plasma PEDF, which is a protein with an apparent molecular weight of 50 kDa, has an amino acid sequence with a high degree of homology or identical to that of human retinal PEDF of SEQ ID No: 1. Human plasma PEDF is specifically recognized by anti-PEDF antibodies and it exhibits at least two of the known biological activities of retinal PEDF: induction of neurite outgrowth in retinoblastoma cells and inhibition of the proliferation of endothelial cells.

According to the present invention, PEDF purified from human plasma has certain biochemical features distinct from those of recombinant human PEDF expressed in mammalian expression systems. In addition, human plasma PEDF exhibits certain biological features distinct from those of recombinant PEDF. In accordance with the present invention, human plasma PEDF is shown to inhibit endothelial cell proliferation at doses at which recombinant PEDF does not exert any inhibitory effect. It is, therefore, disclosed that the plasma PEDF, which is the native form of the protein as it occurs in the human body, is advantageous over recombinant human PEDF.

The present invention further relates to isolated fragments including chemical or enzymatic cleavage fragments of plasma PEDF having at least one biological activity of PEDF. Preferably, the plasma PEDF is obtained from mammalian origin. More preferably, the plasma PEDF is obtained from human sources.

The plasma PEDF may be an impure preparation of PEDF, partially purified PEDF, or highly purified PEDF, and is subjected to at least one cleavage agent to yield PEDF fragments having at least one biological activity of PEDF. A cleavage agent may be a chemical cleavage agent, e.g., cyanogen bromide, or an enzyme, preferably an endopeptidase including, but not limited to, trypsin, chymotrypsin, papain, or V8 protease. PEDF fragments having one or more of the biological activities of PEDF including induction of neurite outgrowth differentiation in retinoblastoma cells, inhibition of endothelial cell proliferation, anti-angiogenic activity and/or promotion of neuron survival are encompassed in the present invention. Plasma PEDF may be isolated by one or more of the purification methods of the present invention and/or by any protein purification method known in the art. Similarly, PEDF fragments may be isolated by one or more of the purification methods of the present invention and/or by any protein purification method known in the art.

The present invention further provides derivatives of isolated plasma PEDF having at least one biological activity of PEDF. Preferably, PEDF may be obtained from mammalian plasma, and more preferably from human plasma. The isolated PEDF is subjected to derivatization to yield PEDF derivatives having at least one biological activity of PEDF. The isolated plasma PEDF may be an impure preparation of PEDF, partially purified PEDF, or highly purified PEDF. PEDF derivatives include, but are not limited to, phosphorylated or dephosphorylated forms of PEDF, glycosylated or deglycosylated forms, cross-linked PEDF, including dimers, methylated, sulfated, myristylated, hydroxylated, oxidized, reduced, acylated, ADP-ribosylated, amidated, cyclized or iodinated forms are encompassed in the present invention. PEDF derivatives may be purified by one or more of the purification methods of the present invention and/or by any purification method known in the art.

According to another embodiment, the present invention further relates to a pharmaceutical composition comprising as an active ingredient PEDF from plasma, a fragment, a derivative, or a salt thereof having at least one biological activity of PEDF and a pharmaceutically acceptable carrier. Preferably, the plasma is obtained from mammalian origin. More preferably, the plasma is obtained from human sources.

In another embodiment, the invention relates to a process for the production of PEDF, which comprises subjecting plasma to sequential precipitation and chromatography. Preferably, the source for PEDF production is mammalian plasma, and more preferably, the source for PEDF production is human plasma.

In one embodiment, the present invention provides a purification process comprising:
(i) subjecting plasma to protein precipitation, separating the protein precipitate, resuspending the precipitate, and subjecting the resuspended precipitate to ion exchange chromatography;
(ii) detecting the presence of PEDF in eluted fractions of step (i), collecting fractions containing PEDF, and subjecting the pooled fractions to dialysis;
(iii) subjecting the dialysate of step (ii) to affinity chromatography; and
(iv) repeating ion exchange chromatography and affinity chromatography as necessary to obtain homogeneous PEDF.

According to the present invention, subjecting human plasma to sequential precipitation and chromatography yielded homogeneous and pure PEDF. The purification of PEDF from plasma may be performed by the purification process of the present invention and/or by any suitable protein purification method known in the art.

According to another aspect of the present invention, PEDF from plasma may be used in methods of treating diseases associated with endothelial cell proliferation. Preferably, PEDF is obtained from plasma of mammaliari origin, and more preferably PEDF is obtained from human plasma. Some diseases including, but not limited to, age-related macular degeneration, diabetic retinopathy and retinal detachment, and tumors exemplified, but not limited to, retinal tumors and choroidal tumors, have been shown to be associated with angiogenesis. The present invention thus provides a method for treating angiogenesis-related diseases and neuronal diseases comprising administering to a patient in need thereof a therapeutic amount of a pharmaceutical composition comprising isolated plasma PEDF of the invention, a salt, a derivative or a fragment thereof.

According to the present invention it has now been shown that PEDF is present in the plasma of human subjects. Thus, detection of PEDF level in the plasma of an individual may indicate whether the individual is prone to develop an angiogenesis-related disorder including ocular diseases or cancer.

Thus, in a further aspect, the invention relates to a method for diagnosing the presence or incipience of angiogenesis-related disorders in a subject comprising:
(i) measuring the level of PEDF in a blood sample of said individual;
(ii) comparing it to the levels within a normal range of healthy control individuals; and
(iii) evaluating the PEDF levels in said samples, wherein a decrease in the PEDF level in the tested individual in comparison to the PEDF level in the said normal range indicates that said tested individual has a likelihood for developing an angiogenesis-related disorder.

According to a further embodiment, the present invention also relates to isolated PEDF derived from liver cells. Cultures of liver cells are useful for obtaining large quantities of PEDF. According to the present invention, PEDF is the most abundant protein in the conditioned medium of cultured liver cells, particularly of human liver cells. Thus, in another embodiment of the invention, the isolated PEDF is secreted by human liver cells.

The present invention also relates to isolated fragments of PEDF, wherein the PEDF is derived from liver cells and the fragments have at least one biological activity of PEDF. Preferably, the liver cells are human liver cells. The PEDF derived from liver cells may be partially purified or highly purified.

The PEDF is cleaved by at least one cleavage agent to yield PEDF fragments having at least one biological activity of PEDF. The cleavage agent may be a chemical cleavage agent or an enzyme, including trypsin, chymotrypsin, papain, or V8 protease. The isolated PEDF fragments having at least one PEDF biological activity including induction of neurite outgrowth differentiation in retinoblastoma cells, inhibition of endothelial cell proliferation, anti-angiogenic activity and/or promotion of neuron survival are encompassed in the present invention. PEDF isolation from liver cells may be performed by any method disclosed in the invention or by any method known in the art. Similarly, the PEDF fragments may be purified by any method disclosed in the invention or by any method known in the art.

The present invention further provides derivatives of PEDF, wherein the PEDF is derived from liver cells and the derivatives have at least one biological activity of PEDF. Preferably, the liver cells are human liver cells. The PEDF is subjected to derivatization to yield PEDF derivatives having at least one biological activity of PEDF. The isolated EDF may be partially purified PEDF or highly purified PEDF. PEDF derivatives including phosphorylated forms of PEDF, glycosylated or deglycosylated forms, cross-linked PEDF, including dimers, methylated, sulfated, myristylated, or iodinated forms are encompassed in the present invention. PEDF derivatives may be purified by one or more of the purification methods of the present invention and/or by any purification method known in the art.

The present invention further provides a pharmaceutical composition comprising as an active ingredient PEDF derived from liver cells, a derivative, a fragment, or a salt thereof having at least one PEDF biological activity and a pharmaceutically acceptable carrier. Preferably, PEDF derived from human liver cells, a derivative, a fragment, or a salt thereof is used as the active ingredient in the pharmaceutical composition.

Further objects of the present invention are directed to pharmaceutical compositions comprising pharmacologically active PEDF, prepared according to the methods disclosed herein and a pharmaceutically acceptable carrier or diluent for the treatment, prevention or diagnosis of disease and disorders in humans and animals, and provides methods for the treatment of cancer and angiogenesis related diseases therewith.

These and other embodiments of the present invention will be better understood in relation to the figures, description, examples and claims that follow.

### BRIEF DESCRIPTION OF THE FIGURES

FIG. 1 shows the homogeneity of the PEDF isolated from human plasma as determined by protein and immunochemical staining. Human plasma was subjected to a series of purification steps. The resulting pure PEDF was shown to be a homogeneous 50 kDa protein by 7.5% sodium dodecyl sulfate polyacrylamide gel electrophoresis (SDS-PAGE) under reducing conditions and silver staining (lane 1), and by immunoblotting either with antibodies raised against a synthetic peptide (residues 327-343) derived from the amino acid sequence of human PEDF (αPEDF 327-343; lane 3), or with antibodies raised against the denatured human plasma PEDF (αPEDF; lane 4). Immunoblotting of this gel with normal rabbit serum (NRS) (control) did not reveal any protein band (lane 2), illustrating the purity of the preparation.

FIG. 2 shows the tryptic peptide fragments of human plasma PEDF. Human plasma PEDF was subjected to SDS-PAGE under reducing conditions, the gel was Commassie blue stained and the 50 kDa protein was excised from the gel. The gel piece was then subjected to trypsin cleavage followed by mass spectrometry. The amino acid sequence of the 15 tryptic peptide fragments is presented. The fragments were designated SEQ ID No:2 to SEQ ID No: 16.

FIG. 3 shows the specific recognition of human plasma PEDF by anti-PEDF peptide antibodies or by anti-PEDF antibodies. Human plasma was subjected to DEAE-Sephacel® chromatography, followed by heparin-agarose chromatography. A sample of the PEDF enriched fraction was analyzed by 7.5% SDS-PAGE under reducing conditions. Lane 1 depicts a Coomassie blue stained gel of this sample. Lane 2 shows an immunoblot of a similar sample revealed with anti-PEDF peptide antibodies (αPEDF 327-343). Lane 3 shows an immunoblot of a similar sample using the antibodies raised against the denatured human plasma PEDF (αPEDF).

FIG. 4 shows the specificity of the anti-PEDF antibodies, which distinguish between plasma PEDF and various serpins. Plasma PEDF (pl-PEDF), plasminogen activator inhibitor-1 (PAI-1), ovalbumin, α₂-antiplasmin, and antithrombin III (each at 1 μg) were analyzed by 7.5% SDS-PAGE under reducing conditions. Upper panel, a Coomassie blue stained gel of these proteins. Lower panel, an immunoblot of similar samples using anti-PEDF antibodies that were raised against the denatured human plasma PEDF.

FIG. 5 depicts a gel filtration chromatogram of human plasma PEDF. PEDF occurs in human plasma as a monomer with an apparent molecular weight of 50 kDa. A sample (50 μg) of purified plasma PEDF or of recombinant human PEDF (for comparison) was layered on top of Sephacryl S-300 column. The elution profile of PEDF was determined by immunoblotting with anti-PEDF peptide antibodies (final dilution 1:20000), and quantitated by densitometry. The results are presented in arbitrary units. The following M.W. markers were used: blue dextran (V0), thyroglobulin (670 kDa), apoferritin (443 kDa), catalase (210 kDa), and bovine serum albumin (67 kDa).

FIG. 6 shows the neurite outgrowth in human retinoblastoma Y-79 cells after pretreatment with human plasma PEDF. Y-79 cells were incubated with 10 nM PEDF (either purified from human plasma (B) or recombinant (C)) in MEM supplemented with 2 mM L-glutamine, antibiotics, and 0.1% insulin, transferrin and selenium mixture. After 7 days in culture, the cells were transferred onto poly-D-lysine coated plates, and their morphology and differentiation state was monitored by light microscopy at various periods of time. The Y-79 morphology at 9 days post-attachment is shown. Cells that were incubated without PEDF (control) are shown in FIG. 5A.

FIG. 7 depicts the inhibition of endothelial cell proliferation by human plasma PEDF. Upper panel, human umbilical vein endothelial cells (HUVEC) were incubated with increasing concentrations of human plasma PEDF in the presence of [³H] thymidine. After 48 hrs the cells were harvested, and the [³H] thymidine incorporation was measured in a scintillation counter. The results presented are from an experiment conducted in triplicates at least 3 times. Lower panel, bovine endothelial cells (BAEC) or fibroblasts were incubated in the absence or presence of plasma PEDF (at 10 or 100 nM). Results are presented as the mean ± SEM of triplicates of 5 experiments.

FIG. 8 shows the secretion of PEDF from hepatoma cells. Subconfluent mouse endothelial cells (H5V), bovine capillary endothelial cells (EC), or human hepatocellular carcinoma cells (HepG2) were grown in serum-free medium for 2 days. The media (60 ml) were collected, concentrated by ammonium sulfate precipitation, and the resulting pellets were resuspended in 20 mM Tris HCl, pH 7.5 (each in 5 ml). A sample (60 μl) from each of the resuspended pellets was subjected to 7.5% SDS-PAGE under reducing conditions. Upper panel, A Coomassie blue stained gel of the samples obtained from the aforementioned media. A lane of M.W. markers is run alongside. Lower panel, An immunoblot of similar samples using the anti peptide antibodies (final dilution 1:10000). PEDF purified from human plasma is also shown for reference.

FIG. 9 shows the CK2 phosphorylation of PEDF under in vitro conditions. Recombinant PEDF (rPEDF) and plasma PEDF (plPEDF) were incubated with CK2 holoenzyme, [γ³²P]ATP, and increasing concentrations of ploy-L-lysine. As a control rPEDF and pIPEDF were incubated with [γ³²P]ATP in the absence of CK2. After 45 min at 22° C. the reaction was arrested by boiling for 5 min in Laemmli's sample buffer. Samples were subjected to 10% SDS-PAGE under reducing conditions. The gel was stained with Coomassie blue (panel B), dried, and subjected to autoradiography (panel A).

FIG. 10 shows trypsin digestion of recombinant PEDF and CK2 phosphorylated PEDF. Panel A, Recombinant PEDF was digested with trypsin. At the indicated time periods, aliquots were removed from the reaction mixture and were centrifuged. The supernatants were collected and Laemmli sample buffer was added. Samples were boiled and subjected to 12.5% SDS-PAGE followed by silver stain. Panel B, Recombinant PEDF was phosphorylated by CK2. The reaction was allowed to proceed for 45 min at 22° C. and was followed by G25 Sephadex column to remove the excess of [γ³²P]ATP. The eluted fraction was then subjected to trypsin digestion as described in Panel A. Samples were subjected to 12.5% SDS-PAGE followed by autoradiography.

FIG. 11 shows the activation of ERK/MAPK by recombinant PEDF and by human plasma PEDF in human umbilical vascular endothelial cells (HUVEC) and in bovine aorta endothelial cells (BAEC). BAEC (panel B), and HUVEC (panel A) were serum-starved for 16 h and then stimulated with rPEDF and pIPEDF for the indicated times. Panel B: dose response of ERK activation by pIPEDF and rPEDF. Cytosolic extracts (30 μg) were subjected to immunoblotting with anti-active doubly phosphorylated ERK antibody (α-pERK, upper panel) or with anti-general ERK1 antibody (α-gERK1, lower panel). The positions of ERK2 and ERK1 are indicated.

FIG. 12 shows the identification of PEDF in plasma from different animals. Upper panel, Four ml of plasma obtained from rat, rabbit, and mouse were purified on heparin agarose. Samples (3 μl) were subjected to 7.5% SDS-PAGE under reducing conditions with a lane of M.W. markers run alongside, and the gel was stained with Coomassie blue. Lower panel, Immunoblotting of the samples depicted in the upper panel. Samples of the partially purified plasmas described in the upper panel were immunoblotted, and the PEDF in them was revealed by staining with the anti PEDF-peptide antibodies.

FIG. 13 shows the identification of PEDF in plasma from randomly chosen healthy human subjects as determined by protein and immunochemical staining. Panel A, Four ml of citrated plasma obtained from each of the individual human subjects were purified on a heparin-agarose beads. Samples (15 μl of each of these purified plasmas) were subjected to 7.5% SDS-PAGE under reducing conditions (lanes 1-6) with a lane of M.W. markers run alongside, and the gel was stained with Coomassie blue. Panel B, immunoblotting of the samples depicted in panel A. Samples (15 μl) of the partially purified plasmas described in panel A (lanes 1-6) were immunoblotted, and the PEDF in them was revealed by staining with the anti-PEDF peptide antibodies. Pure plasma PEDF is also shown as a reference (lane 7).

FIG. 14 shows the decrease in plasma PEDF concentration in a murine model of experimental lung metastasis. Upper panel, C57BL/6 mice were inoculated with D122 lung metastasizing cells. Control mice were injected with PBS only. Twenty-eight days later, treated and control mice were sacrificed, their blood was collected and pooled respectively. Plasmas were purified on heparin agarose. Samples (20 μl) were subjected to 7.5% SDS-PAGE under-reducing conditions with a lane of M.W. markers run alongside, and the gel was stained with Coomassie blue. Lower panel, Immunoblotting of the samples depicted in the upper panel. Samples of the partially purified plasmas described in the upper panel were immunoblotted, and the PEDF in them was revealed by staining with the anti PEDF-peptide antibodies.

### DETAILED DESCRIPTION OF THE INVENTION

The present invention relates to isolated pigment epithelium derived factor (PEDF) obtained from plasma. According to the present invention, PEDF occurs in the plasma of various animals, e.g., rat, rabbit and mouse. As there is high homology in the amino acid sequence of human PEDF and that of other species, plasma of other animals, preferably from mammalian source, may be used as a source for human PEDF. In a preferred embodiment, human plasma is used as a source for isolated PEDF.

According to the present invention, human plasma PEDF has an apparent molecular weight of 50 kDa as determined by gel filtration chromatography and by sodium dodecyl sulfate polyacrylamide gel electrophoresis (SDS-PAGE) under reducing conditions. Trypsin cleavage of human plasma PEDF resulted in the recovery and characterization of 15 peptide fragments covering ˜170 of the 418 amino acid residues of the protein. The amino acid sequence of these peptide fragments was found to be identical to the amino acid sequence of human retinal PEDF of SEQ ID: 1. However, the sequence of the other amino acid residues in plasma human PEDF has not been established directly. Thus, the present invention encompasses human plasma PEDF having at least 70% homology to the amino acid sequence of human retinal PEDF, preferably at least 80% homology, more preferably at least 90% homology, and most preferably at least 95% or greater homology. The isolated human plasma PEDF is recognized by antibodies raised against a synthetic peptide derived from the amino acid sequence of human retinal PEDF and by antibodies raised against human plasma PEDF.

According to the present invention the purified human plasma PEDF exhibits at least two of the known biological activities of the retinal PEDF: neurotrophic activity and inhibition of endothelial cell proliferation. Other biological activities that are attributed to retinal PEDF such as neuronotrophic activity, gliastatic activity and anti-angiogenic activity are encompassed in the present invention.

“Neurotrophic” activity is defined herein as the ability to induce differentiation of a neuronal cell population. For example, PEDF's ability to induce differentiation in cultured retinoblastoma cells is considered neurotrophic activity.

“Anti-angiogenic” activity is defined herein as the ability to inhibit the formation of new blood vessels.

“Neuronotrophic” activity is defined herein as the ability to enhance the survival of neuronal cell populations.

“Gliastatic” activity is defined herein as the ability to inhibit glial cell growth and proliferation.

According to the present invention PEDF purified from human plasma has certain biochemical features distinct from those of recombinant human PEDF expressed in mammalian expression systems. It is now disclosed that the phosphorylation of human plasma PEDF by various protein kinases is lower than that of recombinant PEDF. In addition, human plasma PEDF exhibits certain biological features distinct from those of recombinant PEDF. In accordance with the present invention, human plasma PEDF is shown to inhibit endothelial cell proliferation at doses which recombinant PEDF does not exert any inhibitory effect. It is, therefore, disclosed in the present invention that plasma PEDF, which is the native form of the protein as it occurs in the human body, is advantageous over recombinant PEDF.

The present invention relates to isolated plasma PEDF fragments having at least one of the biological activities of PEDF. Preferably, the fragments of PEDF are obtained from mammalian plasma, and more preferably from human plasma. Fragments of plasma PEDF can be produced by subjecting the protein to at least one cleavage agent. A cleavage agent may be a chemical cleavage agent, e.g., cyanogen bromide, or an enzyme, preferably endoproteinase. Endoproteinases that can be used to cleave PEDF include trypsin, chymotrypsin, papain, V8 protease or any other enzyme known in the art, which is known to produce proteolytic fragments. Preferably, insoluble enzymes are used in order to overcome the necessity of removal of the enzyme itself. The enzymatic cleavage is commonly performed at a temperature range of 22-37° C. for a time period of 30 to 60 min, although lower or higher temperatures may be used for different time periods so long as the desired cleavage fragments are obtained.

The proteolytic fragments of PEDF may be prepared from an impure preparation of plasma PEDF, from partially purified plasma PEDF, or from purified and homogeneous plasma PEDF. An impure preparation of plasma PEDF is defined herein as 20-50% purity, partially purified PEDF is defined herein as 50-90% purity and highly purified PEDF is defined herein as 90-100% purity. The purity range is based on any method for protein analysis, including, but not limited to, Commassie brilliant blue staining of polyacrylamide gels or silver staining of polyacrylamide gels.

The proteolytic fragments having at least one of the PEDF activities may be purified by one or more of the purification methods of the present invention and/or by any protein purification method known in the art. The present invention encompasses PEDF proteolytic fragments, which are modified by any derivatization method known in the art exemplified, but not limited to, reduction, oxidation, phosphorylation, glycosylation, methylation, or sulfation.

The present invention relates to plasma PEDF derivatives. Preferably, the plasma is obtained from a mammalian source, and more preferably from human sources. The derivatives of PEDF may be generated from an impure preparation of plasma PEDF, from partially purified plasma PEDF, or from purified and homogeneous plasma PEDF. The purification of plasma PEDF or derivatives thereof may be performed by one or more purification methods of the invention and/or by any protein purification method known in the art.

Typically, the primary amino acid sequence of a protein may be modified by derivatization using sugar moieties (glycosylation) or by other supplementary molecules such as lipids, phosphate, acetyl groups and the like. The primary amino acid structure may also aggregate to form complexes, most frequently dimers. Such modifications are included in the invention so long as the desired activity of the protein is maintained. It is expected that such modifications may quantitatively or qualitatively affect the activity, however, modifications that preserve or improve at least one of the biological activities of PEDF are included in the present invention.

Individual amino acid residues in a protein may also be modified by oxidation, reduction, myristylation, sulfation, acylation, ADP-ribosylation, amidation, cyclization, disulfide bond formation, hydroxylation, iodination, methylation, or other derivatization method known in the art. Such alterations, which do not destroy the PEDF activity, can occur anywhere in a polypeptide, including the peptide backbone, the amino acid side-chains and the amino or carboxyl termini. It will be appreciated that the same type of modification may be present in the same or varying degrees at several sites in the protein. Also, the PEDF may contain many types of modifications. Such modifications may be present on fragments of plasma PEDF. Such modifications are well described in basic texts and in more detailed research literature. Thus, glycosylation derivatives are included within the scope of the present invention. They include derivatives completely lacking glycosylation (unglycosylated) and derivatives having at least one less glycosylated site (deglycosylated) than the native form as well as derivatives in which the glycosylation has been changed.

Similarly, the present invention encompasses derivatives that are lacking phosphorylated amino acids (dephosphorylated) and derivatives that are partially or fully phosphorylated. Phosphorylation of PEDF is performed by incubating plasma PEDF in the presence of different protein kinases known in the art to phosphorylate tvrosine, serine or threonine residues. Examples for such protein kinases are casein kinase 2 (CK2), PKA, or PKC. PEDF derivatives, which are phosphorylated by one or more protein kinases are included in the present invention so long as their PEDF activity is preserved or improved. Dephosphorylation of PEDF may be performed by incubating PEDF in the presence of a protein phosphatase known in the art to reduce or eliminate the phosphate moieties present in the protein. According to the present invention, human plasma PEDF is phosphorylated by CK2 or PKA to a lower extent than recombinant human PEDF expressed in mammalian expression systems. This indicates that plasma PEDF is biochemically distinct from recombinant PEDF as in its native form it apparently retains or contains more phosphorylated amino acid residues.

Typically, proteins that include extensions, truncations or deletions can result in proteins that are functionally equivalent. Accordingly, it is intended that the present invention encompass all amino acid sequences that result in PEDF protein having at least one of the PEDF biological activities.

Minor modifications in the PEDF primary amino acid sequence may result in proteins, which have decreased or increased activity compared to the PEDF described herein. Such modifications may be deliberate, e.g., by chemical or enzymatic modifications, or may be spontaneous. All proteins produced by these modifications are included herein as long as at least one of the PEDF functions is retained.

The precise chemical structure of all proteins depends on a number of factors. As ionizable amino and carboxyl groups are present in a protein, a particular protein may be obtained as an acidic or basic salt, or in neutral form. All such preparations, which retain their activity when placed in suitable environmental conditions are included in the invention.

The present invention provides a pharmaceutical composition for the treatment of angiogenesis-related disease and neuronal diseases comprising as an active ingredient plasma PEDF, a derivative, a fragment or a salt thereof having at least one of the biological activities of PEDF and a pharmaceutically acceptable carrier. Preferably, the plasma PEDF is obtained from a mammalian source, and more preferably, from human sources.

Any suitable pharmaceutically acceptable carrier can be used within the context of the present invention and such carriers are well known in the art. The choice of carrier will be determined, in part, by the particular site to which the composition is to be administered and the particular method used to administer the composition.

Suitable formulations include aqueous and non-aqueous solutions, isotonic sterile solutions, which can contain anti-oxidants, buffers, bacteriostats, and solutes that render the formulation isotonic with the blood or with the intraocular fluid of the intended recipient, aqueous and non-aqueous sterile suspensions and fat emulsions that can include suspending agents, solubilizers, thickening agents, stabilizers, and preservatives. The formulations can be presented in unit-dose or multi-dose sealed containers, such as ampules and vials, and can be stored in a freeze-dried (lyophilized) form requiring only the addition of the sterile liquid carrier, for example, water, immediately prior to use.

In another aspect, the present invention relates to a process for the production of PEDF, which comprises subjecting plasma to sequential precipitation and chromatography. Preferably, the plasma is obtained from mammals, and more preferably from humans.

In a preferred embodiment, the present invention provides a process for purifying PEDF from plasma comprising:
(i) subjecting plasma to protein precipitation, separating the protein precipitate, resuspending the precipitate, and subjecting the resuspended precipitate to ion exchange chromatography;
(ii) detecting the presence of PEDF in eluted fractions of step (i), collecting fractions exhibiting PEDF activity, and subjecting the pooled fractions to dialysis;
(iii) subjecting the dialysate of step (ii) to affinity chromatography; and
(iv) repeating ion exchange chromatography and affinity chromatography) as necessary to obtain a homogeneous preparation of PEDF.

The methods of obtaining plasma devoid of blood cells are well known in the art. In the present invention human citrated plasma was used as a preferred source for PEDF. However, plasma containing additional anti-coagulants, such as dextrose, may also be used for plasma preparation.

According to the present invention, plasma, e.g., citrated human plasma, is subjected to protein precipitation by methods well known in the art exemplified, but not limited to, ammonium sulfate or polyethylene glycol (PEG). The precipitate is resuspended and the protein fraction containing impure PEDF is subjected to ion, e.g., anion, exchange chromatography, for example, with a Q-Sephacel® or a DEAE-Sephacel®.

The presence of PEDF in the eluted fractions may be detected by any suitable method for detecting the presence of PEDF exemplified, but not limited to, SDS-polyacrylamide gel electrophoresis on, for example, 7.5%, 10% or 12.5% w/v SDS polyacrylamide gels followed by protein staining of the gel, immunoprecipitation with anti-PEDF antibodies, radio immunoassay (RIA) using anti-PEDF antibodies, induction of neurite outgrowth in retinoblastoma cells, inhibition of human or bovine endothelial cell proliferation, and/or immunoblotting with anti-PEDF antibodies.

The present invention further provides antibodies immunoreactive with PEDF. These antibodies can be polyclonal antibodies or monoclonal antibodies. Polyclonal antibodies can be produced according to methods known in the art, including vaccinating an animal with PEDF, collecting and purifying the animal's antisera directed against PEDF. Monospecific polyclonal antibodies can also be produced using methods known in the art. Antibodies, which consist essentially of pooled monoclonal antibodies with different epitopic specificities, as well as distinct monoclonal antibody preparations are also encompassed in the present invention. Monoclonal antibodies are made from antigen containing fragments of the protein by methods well known in the art.

The antibodies can be raised against either the native or denatured human PEDF molecule, or against a peptide derived from PEDF. Polyclonal or monoclonal antibodies raised against PEDF of other species or against a peptide derived from such PEDF, for example, bovine or mouse PEDF, may be used as well since the amino acid sequence of human PEDF shows a high degree of homology with PEDF of other species. According to the present invention, polyclonal antibodies were raised against a peptide having the amino acid residues 327-343 of the human PEDF sequence (herein identified as anti P327-343 antibodies).

There are many different labels for antibodies and methods of labeling antibodies known to those of ordinary skill in the art, which can be used in the detection of PEDF in blood samples. Examples of the types of labels, which can be used in the present invention, include enzymes, radioisotopes, colloidal metals, fluorescent compounds, chemiluminescent compounds, and bioluminescent compounds.

The fractions showing PEDF activity are pooled, the buffer is exchanged by methods known in the art including, but not limited to, dialysis or filtration, and the pooled fractions are then subjected to affinity chromatography, for example, with an immobilized lectin, such as concanavalin A or lentil, with immobilized anti-PEDF antibodies or, more preferably, with immobilized heparin, e.g. heparin-agarose.

For further purification, the above steps of protein precipitation, ion exchange and affinity chromatography may be repeated at least once or as many times as necessary in order to obtain the isolated and homogeneous plasma PEDF.

The above-described purification procedures may be used alone or in combination with other purification techniques known in the art. The purification procedures may use other purification techniques known in the art. Such procedures are intended to produce a PEDF protein fraction of the desired purity. Thus, an impure PEDF protein fraction or a partially purified PEDF fraction disclosed in the present invention can be used as the starting material for subsequent purification procedures known in the art The choromatography methods may be conducted by either batch or column chromatography techniques.

According to the present invention, citrated human plasma is subjected to protein precipitation by polyethylene glycol. The precipitate is separated, resuspended, dialysed and applied onto a DEAE-Sephacel® column. The eluted fractions are detected for the presence of PEDF by immunoblotting with the anti P327-343 antibodies, the fractions containing PEDF are pooled, dialysed, and subjected to heparin chromatography. The fractions eluted from the heparin column containing PEDF as detected by immunoblotting with the anti P327-343 antibodies are pooled, dialysed, and subjected to an additional anion exchange chromatography with a Mono-Q column connected to FPLC. The fractions eluted from the Mono-Q column containing PEDF as detected by immunoblotting with anti P327-343 antibodies are pooled and dialysed, thus obtaining the isolated and homogeneous human PEDF with an apparent molecular weight of 50 kDa.

According to another aspect of the present invention, there is provided a method of treating angiogenesis-related diseases and neuronal diseases comprising administering to a patient in need thereof a therapeutic amount of a pharmaceutical composition comprising isolated plasma PEDF of the invention, a derivative, a fragment or a salt thereof Preferably, PEDF is obtained from mammalian plasma, and more preferably from human plasma.

The present invention relates to methods of treating human for angiogenesis-related disease, including ocular disease or cancer. Ocular-related diseases appropriate for treatment using the PEDF of the present invention include, but are not limited to, age-related macular degeneration (AMD), idiopathic (not age-related) macular degeneration, choroidal neovasculatization, corneal neovascularization, neovascular glaucoma, retinopathy of prematurity and other retinal diseases or disorders that cause growth of abnormal retinal vessels such as diabetic retinopathy, retinal vasculitis, retinal detachment and retinal tears. Examples of the types of cancer that may be treated by PEDF of the present invention are retinal tumors and choroidal tumors. Other types of cancer may be treated by plasma PEDF.

The biological activities of PEDF such as neurotrophic activity, neuronotrophic activity, and gliastatic activity have been previously described. It is, therefore, that plasma PEDF, preferably PEDF obtained from mammalian origin, and more preferably human PEDF, can be used effectively in many neuronal diseases and other insults to the CNS (brain and retina), which are typified by death of neurons and overpopulation by glial cells (gliosis). PEDF can be used effectively in these conditions to prolong the life and functioning of the primary neurons and to arrest glial cell growth.

PEDF has uses in the treatment of retinal diseases including, but not limited to, retinoblastoma, and other ocular tumors. In the case of retinal tumors, PEDF induces the tumor cells to display biochemical and phenotypic characteristics of mature neuronal cells (neurotrophic activity). Such changes are identified by a cessation or reduction in the rate of cell division, which leads to tumor regression or a slowing in the rate of tumor growth.

PEDF can be administered alone or in conjunction with other therapeutic modalities. It is appropriate to administer PEDF as part of a treatment regimen involving other therapies, such as surgery, drug therapy, photodynamic therapy, radiation therapy. PEDF is administered by any suitable route of administration including, but not limited to, intravitreal, subretinal, intravenous or intramuscular injection or injected or applied at sites of tumor growth. Treatment with PEDF is effective for retinal tumors such as retinoblastoma, other neuronal tumors such as neuroblastoma, or tumors of non-neuronal origin. The treatment results in a cessation or reduction in the rate of cell division and a concomitant reduction in the rate of tumor growth, which in turn results in tumor regression.

PEDF is administered, alone or in conjunction with other therapeutic modalities, by intravitreal or subretinal injection for the treatment of diseases of the neural retina, retinal pigmented epithelium, and other ocular tissue. Treatment results in enhanced survival and well being of the photoreceptors and other ocular cells, prolonging their functional life span and delaying the onset of impaired vision and ultimate blindness.

PEDF is administered, alone or in conjunction with other therapeutic modalities, by intravenous or intramuscular injection or application or injection at the site of neuronal cell pathology for treating injuries to nerves and pathologies of cells. Treatment results in enhanced survival of nerve cells and promotion of neurite outgrowth and nerve regeneration.

The PEDF is administered alone or in conjunction with compounds, such as polylactic acid, which facilitate a slow, “sustained release” of the PEDF. A therapeutic amount of PEDF is administered until the patient is treated. “Therapeutic” is meant to include the amelioration of angiogenesis-related disease or neuronal disease, and/or the protection, in hole or in part, against further angiogenesis-related disease or neuronal disease. Angiogenesis-related diseases include, but not limited to, cancer, age-related macular degeneration or diabetic retinopathy. One of ordinary skill in the art will appreciate that any degree of protection from, or amelioration of, one of such diseases is beneficial to a patient. The present invention is particularly advantageous in that a therapeutic amount of

PEDF can be directly applied to affected areas without harmful side effects. PEDF can be administered using a single or multiple applications within a short time period. For persistent ocular-related diseases, such as age-related macular degeneration and diabetic retinopathy, numerous applications of PEDF may be necessary to achieve a therapeutic effect.

The present invention provides a method for diagnosing an individual for the presence or incipience of angiogenesis-related disease. Blood samples are obtained from an individual suspected of developing or having angiogenesis-related disease. Plasma samples, devoid of blood cells, are prepared as known in the art. The plasma samples may be partially purified, for example, affinity purified. Similarly, plasma samples are prepared from healthy control individuals, preferably from at least 2 individuals, more preferably from at least 5 individuals, and most preferably from at least 10 individuals, to obtain a normal range of plasma PEDF. The level of PEDF is evaluated in said samples by any suitable method for detecting the presence of PEDF including SDS-PAGE and protein staining of the gel, immunoprecipitation with anti-PEDF antibodies, RIA with anti-PEDF antibodies, induction of neurite outgrowth in retinoblastoma cells, inhibition of endothelial cell proliferation, and Western blot assay using anti-PEDF antibodies. Preferably, the evaluation of PEDF level in said sampled is carried out by immunoblotting with anti P327-343 antibodies.

A decrease in the PEDF level in the tested individual in comparison to the PEDF level within the normal range of healthy control individuals indicates that said tested individual has a likelihood for developing an angiogenesis-related disease. Among the pathological disorders related to angiogenesis (neovascularization) are ocular diseases and disorders exemplified by, but not limited to, age-related macular degeneration (AMD), a disease that causes growth of abnormal choroidal veins (called choroidal neovascularization), idiopathic (not age-related) macular degeneration, comeal neovascularization, neovascular glaucoma, retinopathy of prematurity and other retinal diseases or disorders that cause growth of abnormal retinal vessels such as diabetic retinopathy, retinal vasculitis, retinal detachment and retinal tears. In addition, tendency of an individual to develop a type of cancer, such as retinal tumors and choroidal tumors or other metastatic tumors that require neovascularization for the growth of the tumors, can be tested according to the present invention by measuring the plasma PEDF level. The present test can thus be used in particular to screen patients with age-related macular degeneration, diabetic retinopathy and metastatic cancers by testing whether they have abnormal blood levels of PEDF.

According to the present invention, it was found that PEDF, originally discovered to act in the eye, occurs in the plasma of normal human subjects at levels of about 8 μl.

According to the present invention, PEDF occurs in plasma of various species. The source of plasma PEDF was suspected to be either the liver as it liver is known that the liver produces and secretes many plasma proteins, or endothelial cells that line the vasculature. It is disclosed in the present invention that cultures of liver cells are useful for obtaining large quantities of PEDF The present invention thus relates to isolated PEDF derived from liver cells. According to the present invention PEDF is the most abundant protein in the conditioned medium of cultured liver cells, particularly of human liver cells. Thus, in a preferred embodiment of the invention, the isolated PEDF is derived from human liver cells.

The present invention also relates to isolated fragments of PEDF, wherein the PEDF is secreted by liver cells and the fragments having at least one of the biological activities of PEDF. The PEDF may be partially purified or highly purified. The PEDF secreted by liver cells is cleaved by at least one cleavage agent to yield PEDF fragments having at least one of the biological activities of PEDF. Preferably, the liver cells are human liver cells. The cleavage agent may be a chemical cleavage agent, e.g., cyanogens bromide, or an enzyme, including trypsin, chymotrypsin, papain, or V8 protease. The isolated PEDF fragments having at least one of the PEDF biological activities including induction of neurite outgrowth differentiation in retinoblastoma cells, inhibition of endothelial cell proliferation, anti-angiogenic activity and/or promotion of neuron survival are encompassed in the present invention. The isolation of PEDF from liver cells may be performed by any method disclosed in the invention or by any method known in the art. Similarly, the PEDF fragments may be purified by any method disclosed in the invention or by any method known in the art.

The present invention further provides derivatives of isolated PEDF, wherein the isolated PEDF is secreted from liver cells and the derivatives having at least one of the biological activities of PEDF. Preferably, the isolated PEDF is secreted from human liver cells. PEDF isolation from liver cells may be performed by any method disclosed in the invention or by any method known in the art. The derivatives of PEDF are exemplified, but not limited to, glycosylated or deglycosylated forms, cross-linked PEDF, including dimers, methylated, sulfated, myristylated, iodinated, phosphorylated or other modified derivatives, e.g., nitrated derivatives in which the tyrosine residues are nitrated.

The present invention further relates to a pharmaceutical composition for the treatment of angiogenesis-related diseases and neuronal diseases comprising a pharmaceutically acceptable carrier and, as active principle, an effective amount of PEDF secreted by liver cells, a derivative, a fragment or a salt thereof having at least one of the biological activities of PEDF. Preferably, the PEDF secreted by human liver cells is the active principle in the pharmaceutical composition.

The invention will now be illustrated by the following non-limiting examples.

### EXPERIMENTAL

(a) Materials

DEAE-Sephacel, Sepharose S-300, enhanced chemiluminiscent (ECL) western blotting detection reagents, and [³H] Thymidine were purchased from Amersham Pharmacia Biotech. Polyethylene glycol (PEG) MW 3350, α2 antiplasmin, antithrombin III, heparin-agarose, and horseradish peroxidase conjugated anti rabbit IgG were purchased from Sigma. Inject maleimide activated keyhole limpet haemocyanin (KLH) was purchased from Pierce. Plasminogen activator inhibitor-1 (PAI1) was purchased from Biopool AB. The ITS mix containing 5 μg/ml insulin, 5 μg/ml transferrin, and 5 μg/ml selenium was purchased from Gibco BRL. The solution of vitamins that were added to the Minimum Essential Medium (MEM) was purchased from Biological Industries, Beit Haemek. Ovalbumin was purchased from Pharnacia. All other materials were obtained from the best available commercial source.

(b) Preparation of antibodies against denatured PEDF (anti-PEDF) and against the peptide 327-343 of bovine PEDF (anti P327-343)—Antibodies against denatured PEDF were raised in rabbits by subcutaneous injections of the 50 kDa protein band, excised from a 7.5% SDS-PAGE (run under reducing conditions). A peptide having the amino acid residues 327 to 343 of bovine PEDF (disclosed in Wu et al., 1995) was synthesized by solid-phase synthesis, purified using reverse phase HPLC (C-18), and analyzed by its amino acid composition. The peptide was bound to an Imject maleimide activated KLH, and injected subcutaneously to rabbits. A standard immunization protocol was utilized for the preparation of the two antibodies (Harlow et al., 1988). Purification of the IgG fraction was conducted as previously described (Harlow et al., 1988).

(c) SDS-PAGE and immunoblotting—PEDF was subjected to a 7.5% SDS-PAGE under reducing conditions using the buffer system of Laemmli (Laemmli U., 1970). Where indicated, immunoblotting was carried out using rabbit antibodies raised against either the denatured PEDF molecule, herein anti-PEDF antibodies (final dilution 1:1000, or as otherwise indicated), or against the synthetic peptide derived from the PEDF amino acid sequence, herein anti P327-343 antibodies or αPEDF 327-343 (final dilution 1:10000, or as otherwise indicated). The procedure was followed by an incubation of the nitrocellulose paper with a goat anti-rabbit IgG conjugated to a horseradish peroxidase (final dilution 1:2500). PEDF was visualized by the ECL detection method.

(d) In gel proteolysis and mass spectrometry analysis—Following the SDS-PAGE of PEDF samples, the stained protein bands in the gel were cut out, reduced with 10 mM dithiothreitol (DTF), and the sulfhydryl groups were blocked with 100 mM iodoacetamide in 10 mM ammonium bicarbonate. The gel pieces were treated with 50% acetonitrile in 10 mM ammonium bicarbonate (to remove the stain), dried, and rehydrated with 10 mM ammonium bicarbonate containing about 0.1 μg trypsin per sample (overnight at 37° C.). The resulting peptides were extracted with 60% acetonitrile containing 0.1% trifluoroacetate, and resolved by reverse-phase chromatography on a 1×150 mm Vydac C-18 column (linear gradient of 4-65% acetonitrile in 0.025% trifluoroacetate, at 1%/min and a flow rate of 40 μl/min). The sample was electrosprayed into an ion-trap mass spectrometer (LCQ, Finnigan, San Jose, Calif.). The mass spectrometry was performed in the positive ion mode using repetitively full MS scan, and collision induced dissociation (CID) of the most abundant ions selected from the first MS scan. The spectra were compared with the simulated proteolysis and CID pattern of the proteins in the “genpept” using the Sequest software (J. Eng and J.Yates, University of Washington and Finnigan, San Jose). The amino terminal of the protein was sequenced on a Peptide Sequencer 494A (Perkin Elmer).

(e) Stable expression of PEDF in CHO cells—The plasmid pCEP4-PEDF, containing the human full length cDNA (kindly provided by Dr. N. P. Bouck, The Picower Institute for Medical Research, Manhasset, N.Y.) was introduced into CHO cells using the LipofectAMINE reagent (Life Technologies, Inc.) following the manufacturer's instructions. Stable transfected cells were selected by their resistance to 500 μg/ml hygromycin B. The stable transfectants were grown to confluence, and then cultured in serum free medium. After 3 days the medium was collected, and the recombinant PEDF was purified on a Chelating Sepharose Fast Flow (Pharmacia Biotech) following the manufacturer's instructions.

(f) Cell cultures—Bovine aorta endothelial cells (BAEC) were cultured in DMEM (glucose concentration 1 g/liter) supplemented with 10% (v/v) heat inactivated calf serum (HyClone) and antibiotics. Fibroblasts 3T3 Balb/C were grown in DMEM (glucose concentration 4.5 g/liter) supplemented with 10% (v/v) heat inactivated fetal calf serum (Gibco BRL), and antibiotics. Human umbilical vascular endothelial cells (HUVEC) were grown on gelatin (0.2%) coated plates in M-199 supplemented with 20% fetal calf serum (Gibco BRL), antibiotics and MEM-vitamins. Xenopus basic FGF (2 ng/ml; kindly provided by Dr. G. Neufeld, The Technion, Israel Institute of Technology, Israel) was added every other day. All the cells were grown in a humidified incubator (37° C.) with an atmosphere containing 5% CO₂.

(g) In vitro phosphorylation of PEDF by CK2—The phosphorylation assay (40 μl) contained the following constituents at the final concentration: recombinant PEDF (rPEDF), human plasma PEDF or the various rPEDF mutants 50 μg/ml, CK2 4 μg/ml, glycerol 2%, sodium chloride 20 mM, β-mercaptoethanol 0.1 mM, magnesium chloride 20 mM, ATP 10 μM (3 Ci/mmol), ploy-L-lysine 200 nM, and Tris-HCl 50 mM pH 7.4. The phosphorylation reaction was allowed to proceed for 45 minutes at 22° C.

(h) Trypsin cleavage of rPEDF—The reaction mixture (300 μl) contained the following constituents at the indicated final concentrations: rPEDF 100 μg/ml and rPEDF phosphorylated by CK2 10 μg/ml dialyzed against reaction buffer (Tris-HCl 20 mM pH 7.5, sodium chloride 150 mM, EDTA I mM), and insoluble trypsin (7 U/ml). The reaction was allowed to proceed at room temperature, under gentle agitation. At selected time points (10, 30 and 60 minutes) aliquots (60 μl) were transferred to conical tubes and were centrifuged. The supernatants were collected and Laemmli's sample buffer was added. The samples were boiled for 5 minutes and subjected to 12.5% SDS-PAGE followed by silver stain or autoradiography.

(i) Activation of MAPK cascades—BAEC and HUVEC cells were grown in 10 cm plates and were serum starved (0.1% CS and 1% FCS respectively) for 16 hr. The cells were then treated with various stimuli for the indicated times. Following stimulation, cells were washed twice with ice-cold PBS and once with ice-cold buffer A (50 mM glycerophosphate, pH 7.3, 1.5 mM EGTA, 1 mM EDTA, 1 mM dithiothreitol, 0.1 mM sodium vanadate). Cells were harvested in 0.3 ml buffer H: buffer A containing 1 mM benzamidine, 10 μg/ml aprotinin, 10 μg/ml leupeptin, 2 μg/ml pepstatin A, followed by sonication (2 pulses for 7 sec of 50 W), and centrifugation (20,000×g, 15 min, 4° C.). The supernatants, which contained cytosolic proteins were collected, and aliquots (30 μg) were separated on 10% SDS-PAGE, followed by Western blotting with mouse monoclonal anti-active MAPKs (ERK, JNK, p38MAPK) (Sigrna, Israel). Total MAPKs were detected with polyclonal antibodies for the various MAPKs as a control (Sigma, Israel). The blots were developed using alkaline phosphatase or horseradish peroxide-conjugated anti-mouse or anti-rabbit antibodies (Jackson ImmunoResearch Laboratories, Inc., PA).

### EXAMPLE 1

### Purification of PEDF from Human Plasma

All the procedures for PEDF purification were carried out at 4° C. Human citrated plasma (I liter; obtained from the Chaim Sheba Medical Center, Israel) was cooled on ice. The following additives were added (final concentrations are given): reduced glutathione (1 mM), benzamidine hydrochloride (10 mM), and para methylsulfonylfluoride (PMSF) (1 mM). The citrate ions were removed by precipitation with barium chloride as follows: 80 ml of BaCl₂ were added, the mixture was stirred for 1 hr, and then the barium citrate was removed by centrifugation (6000 g for 15 min). The supernatant was collected, and 220 ml of 50% PEG 3350 in 30 mM Tris-HCl, pH 7.4, containing 0.1 M NaCl were added (final concentration of PEG 9%). The mixture was stirred for 1 hr, centrifuged (6000 g for 15 min), and the resulting precipitate was discarded. A volume of 315 ml of 50% PEG 3350 in 30 mM Tris-HCl, pH 7.4, containing 0.1 M NaCl, was added to the plasma supematant (final concentration of PEG 20%). The mixture was stirred again for 1 hr, and then centrifuged as above. The 9-20% PEG precipitate was dissolved in 20 mM sodium phosphate and 2 mM EDTA, pH 7.0, containing reduced glutathione (1 mM), benzamidine hydrochloride (2 mM), and PMSF (1 mM), and then applied onto a DEAE-Sephacel® column (2.9×40 cm). The column was washed with 600 ml of the aforementioned buffer containing also 25 mM NaCl, and then the proteins were eluted with a linear gradient of NaCl (0.025-0.3 M), 1 L in each vessel. The presence of PEDF was determined by immunoblotting, using the anti P327-343 antibodies (final dilution 1:10000).

The fractions containing PEDF (˜70 ml) were pooled, dialyzed against 20 mM sodium phosphate, pH 6.5, containing 20 mM NaCl (buffer H), and then added to heparin agarose beads (7 ml). The suspension was agitated for 30 min, and then transferred onto a column (1.2×7 cm). This column was washed with 30 ml of buffer H, and the proteins were then eluted stepwise with 0.1, 0.2, 0.3, 0.4, and 0.5 M NaCl in buffer H (2 ml/fraction; 3 fractions for each NaCl concentration). The fractions that contained PEDF (identified by immuno-blotting using anti P327-343 antibodies), were pooled (˜20 ml), dialyzed against 20 mM Tris-HCl, pH 7.4, and applied onto a Mono Q-FPLC column (HR 5/5, 1 ml, Amersham Pharmacia Biotech), which was connected to an HPLC system (Hewlett Packard 1050) with a UV detection at 280 nm. The elution was conducted with a linearly increasing concentration of NaCl up to 0.3 M (flow rate, 0.5 ml/min). Fractions were collected, and the presence of PEDF was determined by immunoblotting using the anti P327-343 antibodies.

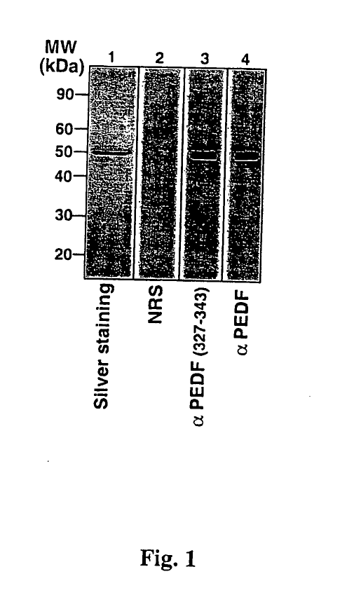

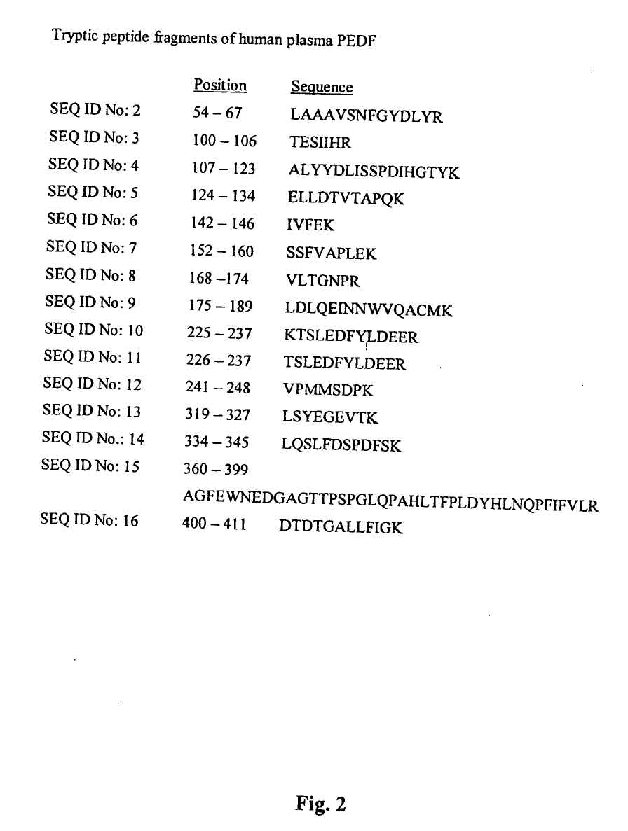

As seen in FIG. 1, this series of purification steps resulted in the isolation of a homogeneous protein (M.W. ˜50 kDa) as revealed by silver staining after SDS-PAGE (FIG. 1, lane 1). When the 50 kDa band was subjected to mass spectrometry, fifteen peptides were obtained. FIG. 2 lists the amino acid sequence of these peptide fragments (SEQ ID No. 2 to SEQ ID No.16) and indicates that the protein isolated from human plasma is indeed PEDF, a protein previously shown to be produced in the retinal pigment epithelial cells, and to take part in neovascularization of the retina (Steele et al., 1993). The final concentration of PEDF purified from plasma was usually ˜40 μg/ml.

### EXAMPLE 2

### Characterization of Human Plasma PEDF

In order to characterize human plasma PEDF by immunological and biochemical means, the homogeneous PEDF obtained in Example 1 (FIG. 1, lane 1) was subjected to immunoblotting with either anti P327-343 antibodies or with anti-PEDF antibodies. As shown in FIG. 1, both the anti P327-343 antibodies (α PEDF 327-343) and the anti-PEDF antibodies (α PEDF) specifically recognized the PEDF purified from human plasma (FIG. 1, lanes 3 and 4, respectively).

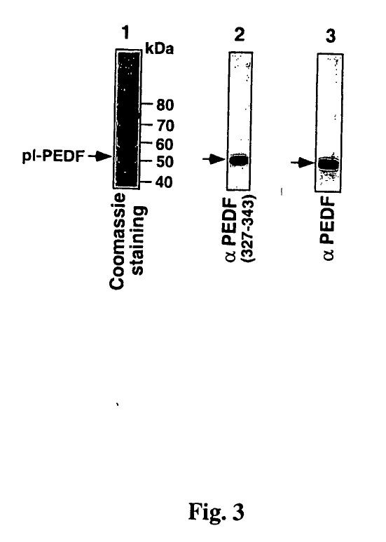

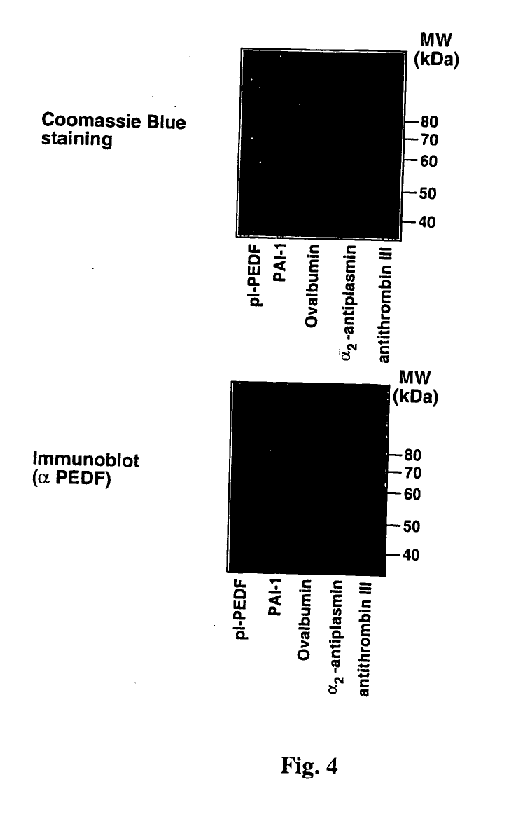

The specific recognition of PEDF by these two polyclonal antibodies is also shown in FIG. 3. Human plasma was subjected to a partial purification by DEAE-Sephacel® chromatography, followed by heparin-agarose chromatography. A sample of the PEDF enriched fraction was analyzed by 7.5% SDS-PAGE under reducing conditions followed by immunoblotting with either anti P327-343 antibodies or with anti-PEDF antibodies. As shown in FIG. 3, both antibodies recognized PEDF within a large repertoire of plasma proteins. These antibodies also distinguished between PEDF and various serpins. As shown in FIG. 4, the anti-PEDF antibodies specifically recognized plasma PEDF but failed to recognize several proteins belonging to the serpin superfamily, such as PAI-1, ovalbumin, α2 antiplasmin, and antithrombin III.

The characterization of the oligomeric state of plasma PEDF was carried out by gel filtration. PEDF was layered on top of a 1.6×120 cm Sephacryl S-300 column equilibrated and run with 50 mM Tris-HCl, pH 7.4, containing 0.1% polyethylene glycol 3350. The column was run at a flow rate of 15 ml/hr, and 1 ml fractions were collected. The elution profile of PEDF was determined by immunoblotting with anti P327-343 antibodies.

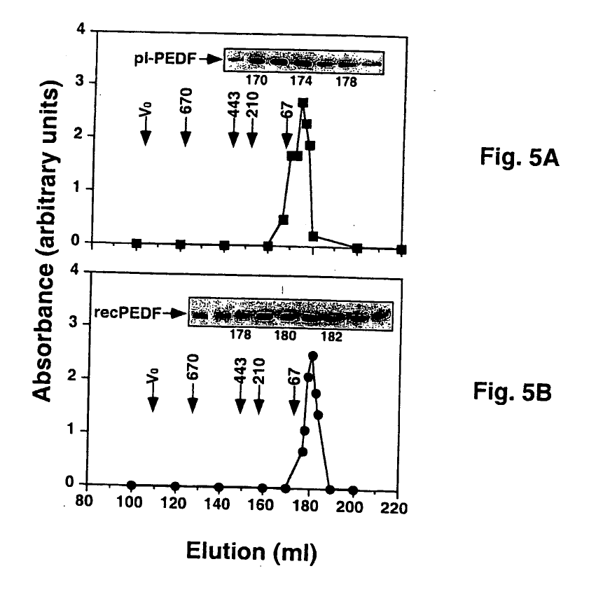

As depicted in FIG. 5A, the PEDF isolated from human plasma was found to be a monomer with an apparent molecular weight of 50 kDa. As shown in FIG. 5B, a similar elution profile was obtained with the human recombinant PEDF expressed in CHO cells prepared as in Section (e) above, and used herein as a control.

### EXAMPLE 3

### Biological Activity of Human Plasma PEDF

PEDF isolated from the culture medium of human retinal pigment epithelial cells was shown previously to induce retinoblastoma cell differentiation (Steele et al., 1993). In order to find out whether plasma PEDF can induce such differentiation, a neurite outgrowth assay in retinoblastoma cells was conducted as follows:

Two ml of a human Y-79 retinoblastoma cell suspension (obtained from ATCC; 2.5×10⁵ cells/ml) were incubated with 10 nM PEDF (either purified from human plasma or recombinant) in MEM supplemented with 2 mM L-glutamine, antibiotics, and 0.1% ITS. After 7 days in culture the cells were transferred to poly-D-lysine coated plates, and their neurite outgrowth was monitored by light microscopy at various periods of time.

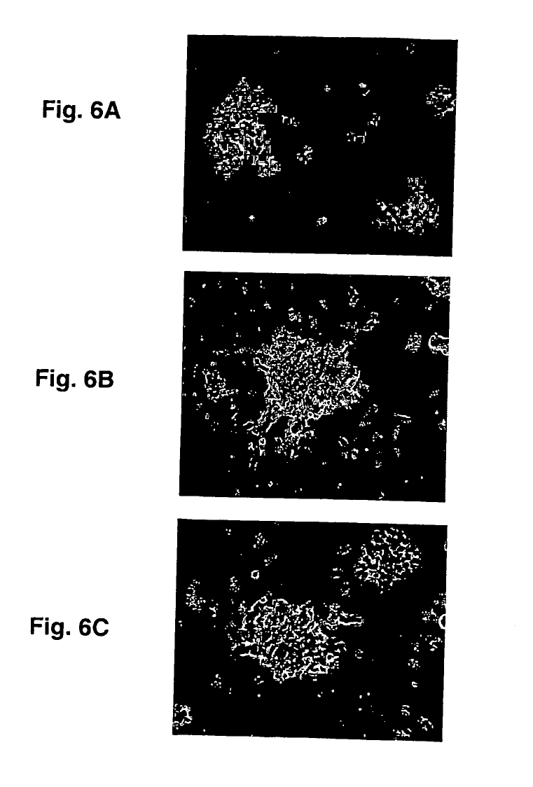

As shown in FIG. 6A, untreated retinoblastoma cells are round shaped cells that grow in aggregates. Treatment of these cells with purified human plasma PEDF induced neurite outgrowth (FIG. 6B). Recombinant PEDF expressed in CHO cells (as described in Section (e) above), at an identical concentration of 10 nM, induced similar neurite outgrowth and served herein as a control (FIG. 6C).

PEDF was previously shown to inhibit cell proliferation of endometrial carcinoma cells. In order to determine whether human plasma PEDF can exert similar inhibitory activity, a cell proliferation assay was conducted as follows:

Confluent HUVEC, BAEC or fibroblasts were trypsinized, seeded in 24 well plates (25×10³ cells/well in 0.5 ml for HUVEC, or 15×10³ cells/well in 0.5 ml for BAEC or fibroblasts), and grown until they reached subconfluence (2 to 3 days in culture). The medium for BAEC and fibroblasts was replaced by a fresh serum free medium supplemented with antibiotics, and the incubation was continued for 2 additional days. Thereafter, the medium was aspirated, and the cells were treated with PEDF in a serum free medium containing antibiotics, and [³H] thymidine (2 μCi/ml; 0.3 ml/well). In the case of HUVEC, the cells were treated with PEDF in medium containing 10% serum, bFGF (2 ng/ml), and [³H] thymidine (2 μCi/ml; 0.3 ml/well). After 2 days all the cells were washed with ice-cold PBS (3 washes), and fixed (30 min at 4° C.) with trichloroacetic acid (0.5 ml; 0.1%). The acid was aspirated, an ethanol solution (0.5 ml; 95%) was added, the wells were air dried (5 min), and finally 1 M NaOH (0.4 ml) was added. The samples (0.3 ml) were then counted in a β-scintillation counter.

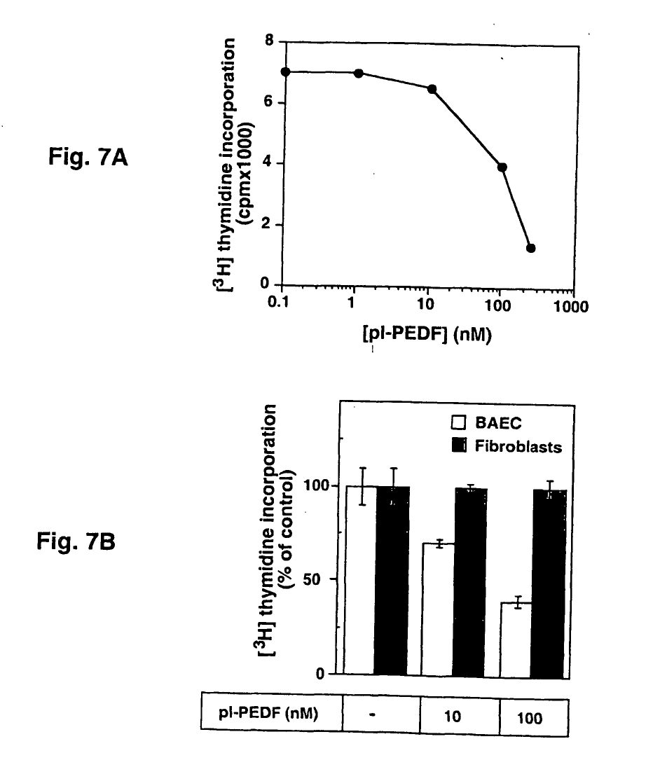

As seen in FIG. 7, purified human plasma PEDF was found to inhibit the proliferation of vascular endothelial cells, e.g., human umbilical vein endothelial cells or bovine aorta endothelial cells (FIGS. 7A and 7B). This inhibition was cell specific since human plasma PEDF did not exert any inhibitory activity on fibroblasts (FIG. 7B).

The anti-angiogenic activity of plasma PEDF is checked by any method known in the art for testing inhibition of neovascularization such as neovascularization in rat cornea or neovascularization in rat aorta ring. In the case of the rat cornea model, plasma PEDF, PEDF derivatives or PEDF fragments are incorporated with vehicle (PBS) into hydron pellets, which are implanted into the avascular rat cornea. The ingrowth of vessels from the limbus toward the hydron pellet after 7 days is then detected. Massive ingrowth without addition of plasma PEDF is used as a control.

### EXAMPLE 4

### Cultured Human Liver Cells Secrete PEDF to the Medium

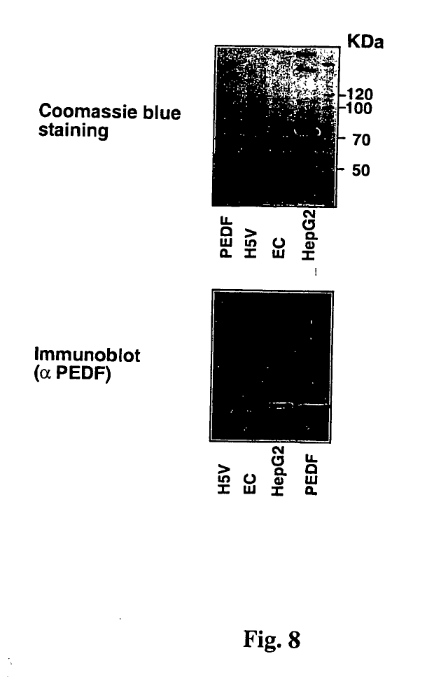

In order to establish the source of PEDF in plasma, a few cell types, such as endothelial cells that line the blood vessels, or hepatocytes that are known to synthesize and secrete many blood proteins, were tested for their capability to secrete PEDF. FIG. 8 shows that hepatoma cells (HepG2) were found to secrete to their medium a 50 kDa protein, which cross-reacted with the anti PEDF-peptide antibodies. Similar results were also obtained with the anti denatured PEDF antibodies (not shown). Other cell types, such as mouse or bovine endothelial cells did not secrete PEDF to their culture medium (FIG. 8). These results indicate that liver cells, or more specifically hepatoma cells such as HepG2, may be used as a source for PEDF.

### EXAMPLE 5

### PEDF Purified from Plasma is not Identical to Recombinant PEDF

Analysis of the primary sequence of PEDF revealed the existence of several putative phosphorylation sites for protein kinase 2 (CK2). Protein kinase CK2 is a holoenzyme composed of two regulatory β-subunits and two catalytic α- or α′-subunits. It is a kinase that is not responsive to known second messenger molecules, but it can be moderately activated by polyamines (such as spermine and spermidine) and by polylysine.

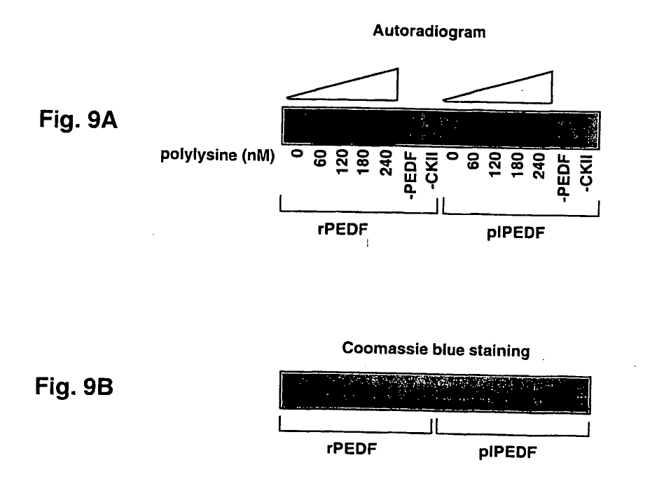

The possibility that PEDF is a substrate of CK2 in vitro was first tested. Recombinant PEDF (rPEDF) and PEDF purified from human plasma (plPEDF) were incubated with the holoenzyme and [γ³²P]ATP, with an increasing concentration of poly-L-lysine. Phosphorylated products were analyzed by 10% SDS-PAGE, which was followed by coomassie blue staining and autoradiography. FIG. 9A shows that both rPEDF and pIPEDF were phosphorylated by CK2 in the presence of poly-L-lysine. The effect of poly-L-lysine varied according to its concentration, being an absolute requirement for PEDF phosphorylation. It is important to note that CK2 phospborylation of rPEDF was much more significant compared with pIPEDF. This difference was not due to different amounts of PEDF since an equal amount of substrate was present in each treatment (FIG. 9B).

To further characterize the in vitro CK2 phosphorylation of PEDF, the stoichiometry of this phosphorylation was determined. When rPEDF was subjected to phosphorylation by CK2 in the presence of [γ³²P] ATP, incorporation of ³²P occurred with a stoichiometry of ˜0.1 mol of phosphate per mol of rPEDF. When rPEDF was heat-treated (15 min at 56° C.) prior to the phosphorylation (a treatment that opens up the PEDF molecule and exposes potential phosphorylation sites), the stoichiometry of phosphorylation was increased, and reached a plateau at ˜0.25 mol/mol. We also checked the phosphorylation of human plasma PEDF under the same conditions. When plasma PEDF was subjected to phosphorylation by CK2 in the presence of [γ³²P] ATP, incorporation of ³²P occurred with a stoichiometry of ˜0.02 mol of phosphate per mol of plasma PEDF. When plasma PEDF was heat-treated (15 min at 56° C.) prior to the phosphorylation, the stoichiometry of phosphorylation was increased, and reached a plateau at ˜0.12 mol/mol. While heat treatment of rPEDF increased the stoichiometry by 2.5 folds, that of plasma PEDF increased by 6 folds.

The fact that CKII phosphorylates plasma PEDF at relatively low stoichiometry compared to rPEDF indicates that PEDF in plasma is most likely more pre-phosphorylated and is present in vivo as a phosphoprotein. These results, therefore, indicate that rPEDF is not identical in its biochemical features to the native form of PEDF purified from human plasma.

CK2 phosphorylates serines and threonines immersed in acidic sequence within proteins and peptides. The minimum requirement for CK2 phosphorylation is depicted by the sequence S/T-X-X-D/E. However, the presence of additional Asp or Glu residues at positions −3, +1, +2, +4, +5, or +7 (relative to the Ser or Thr residues to be phosphorylated) improves the phosphorylation efficacy. Most of the physiological substrates of CK2 have at least one additional acidic amino acid residue in one of these positions. By scanning the primary sequence of PEDF for potential phosphorylation sites, 11 putative sites came up that meet with the consensus sequence requirements: S24, S114, T121, S195, T219, T226, S227, T287, S328, S336, and T354. Of these, S24,S114, S195, T226, S227 and T287 were considered as preferred targets for phosphorylation since they contain additional acidic residues in the preferred positions mentioned above.

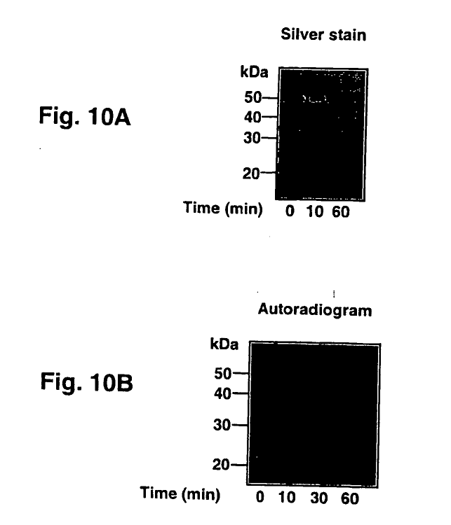

In an attempt to identify the actual CK2 phosphorylation site(s) in PEDF, rPEDF was digested with trypsin. As seen in FIG. 10B, digestion of rPEDF with trypsin yielded two major fragments with an apparent molecular weight of 20kDa and 30 kDa as detected by silver staining of the gel. rPEDF was then phosphorylated by CK2, passed through Sephadex G25 column, and the eluted fraction was digested with trypsin. As seen in the autoradiogram of FIG. 10A, only the 20 kDa fragment was phosphorylated by CK2, implying that the CK2 phosphorylation site is located within the 20 kDa fragment of the trypsin digested rPEDF.

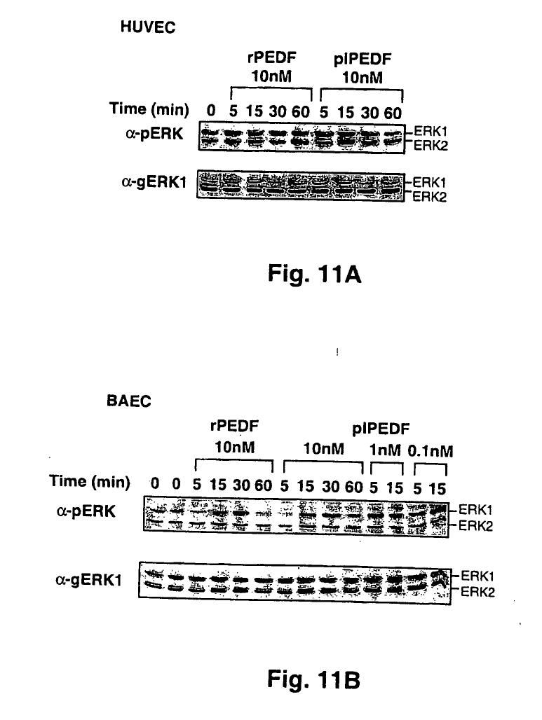

In order to further analyze whether plasma PEDF is identical to rPEDF, the activation of intracellular signaling cascade, specifically that of ERK1/2 (p44/42 MAPKs) by these two proteins was tested. Serum starved endothelial cells were incubated with rPEDF or with plasma PEDF for the time indicated, and cell lysates were then subjected to immunoblotting analysis using antibodies which specifically recognize the doubly phosphorylated active form of MAPKs. As seen in FIG. 11, rPEDF activated ERK in endothelial cells, whether obtained from a bovine source (e.g., BAEC; FIG. 11A) or from a human source (e.g., HUVEC; FIG. 11B). A more pronounced phosphorylation was obtained when human umbilical vascular endothelial cells (HUVEC) were stimulated with PEDF purified from human plasma (FIG. 11A). As seen in FIG. 11A, addition of plasma PEDF to HUVEC for 5, 15, and 30 min resulted in higher. activation of ERK compared to that obtained with rPEDF.

The effect of rPEDF on endothelial cell proliferation was also tested. While plasma PEDF was found to inhibit HUVEC and BAEC proliferation as described in Example 3, rPEDF was totally inactive at these doses. Therefore, these results indicate that plasma PEDF is more active than rPEDF in at least two biological activities tested.

### EXAMPLE 6

### PEDF Occurs in the Plasma of other Animals

In an attempt to establish that PEDF is a natural resident in human blood, the possibility that it occurs in the blood of other mammals was tested.

Four ml of the citrated plasma obtained from the animal were dialysed against buffer H, and then added to heparin agarose beads (0.5 ml of settled volume/each plasma). The suspension was agitated for 30 min at 4° C., and then transferred into a column. Each column was washed with 30 ml of buffer H, and then incubated (10 min) with 0.5 M NaCl in buffer H (0.6 ml). The eluate of each column was dialyzed against 20 mM Tris-HCl, pH 7.4, and then an aliquot (15 μl) was subjected to SDS-PAGE and immunoblotting as described below.

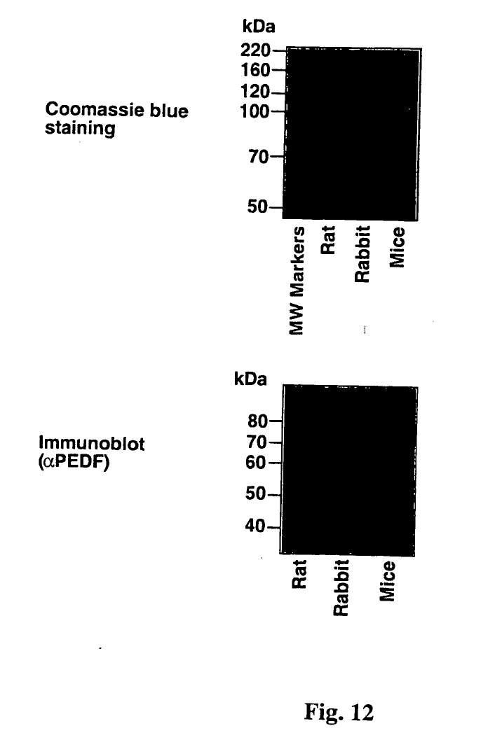

As shown in FIG. 12, a protein similar to PEDF is present in the blood of rat, rabbit, and mouse. It should be noted, however, that the concentration of this protein varied in the different mammals.

### EXAMPLE 7

### Detection of PEDF in Plasma of Healthy Human Subjects

In accordance with the present invention PEDF isolated from human plasma inhibits endothelial cell proliferation. Since angiogenesis in pathological processes involves enhanced endothelial cell proliferation, detection of PEDF level in the plasma of an individual and of a healthy control and evaluation of the decrease in the PEDF level in the tested individual in comparison to the PEDF level of the healthy control, may indicate the susceptibility of said tested individual to develop an angiogenesis-related disorders.

PEDF level was assessed in human plasma by the following method: Four ml of citrated plasma were dialysed against buffer H as described in Example 1, and then added to heparin agarose beads (0.5 ml of settled volume/each plasma). The suspensions were agitated for 30 min at 4° C., and then transferred onto a column (1.2×7 cm each). Each column was washed with 30 ml of buffer H, and then incubated (10 min) with 0.5 M NaCl in buffer H (0.6 ml). The eluate of each column was dialyzed against 20 mM Tris, pH 7.4, and then samples (15 μl) were subjected to SDS-PAGE under reducing conditions.

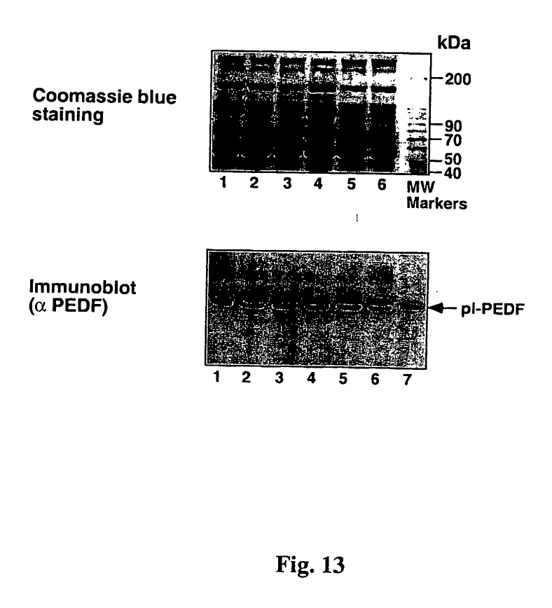

FIG. 13A shows a Coomassie Blue stained gel of plasma samples from 6 healthy human subjects. The level of PEDF in these plasma samples was evaluated by immunoblotting with anti P327-343 antibodies. Samples (15 μl) of the partially purified plasmas were immunoblotted with anti P327-343. As shown in FIG. 13B, the concentration of PEDF in the different plasmas was found to be similar, and was estimated to be ˜8 μg/ml.

### EXAMPLE 8

### Detection of Plasma PEDF in a Murine Model of Experimental Lung Metastasis

The inhibitory activity of PEDF on endothelial cell proliferation is further supported by the findings obtained from rat and mouse models of ischemia-induced retinopathy. In these models, administration of anti-PEDF antibodies to rat corneas induced angiogenesis in these corneas, while systemic administration of PEDF to the mice was shown to prevent retinal neovascularization. Since neovascularization is known to be a major factor in tumor progression, it was suggested that PEDF may control tumor growth. To test this hypothesis, we induced lung metastases in mice and compared the level of their plasma PEDF to that of control healthy mice.

C57BL/6 male mice (8 weeks old) were inoculated intravenously with D122 lung-metastasizing cells (10⁶ cells/mouse; 10 mice) (Popovic et al. (1998). Control mice were injected with phosphate buffer saline only (10 mice). Twenty-eight days later (when the lungs contained metastatic nodules and were 6 fold heavier than healthy lungs), treated and control mice were sacrificed, their blood was collected and pooled respectively. The detection of PEDF in the plasma of mice was conducted as described in example 7.

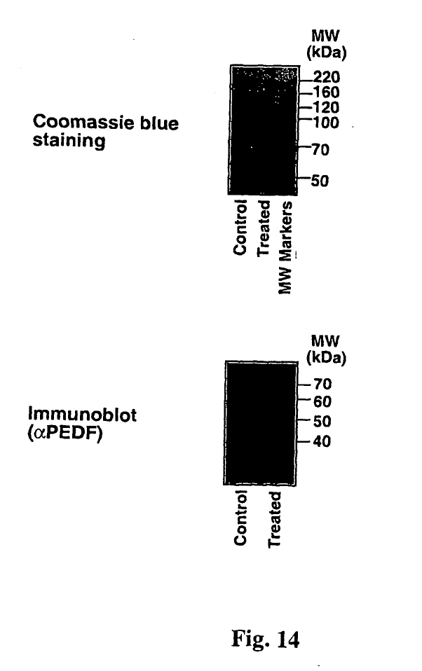

As shown in FIG. 14, mice bearing lung metastases had lower level (˜30%) of plasma PEDF than control mice as determined by densitometry analysis of the PEDF band. These findings indicate that in the course of lung metastasis progression the level of plasma PEDF is decreased, raising the possibility that a decrease in plasma PEDF may promote tumor growth.

The effect of administration of exogenous PEDF from plasma on tumor growth is tested in tumor bearing animals using various routes of administration and various dosing schedules with increasing dose levels.

It will be appreciated by persons skilled in the art that the present invention is not limited by what has been particularly shown and described herein above. Rather the scope of the invention is defined by the claims that follow.

References

Dawson, D. W., Volpert, O. V., Gillis, P., Crawford, S. E., Xu, H., Benedict, W., and Bouck, N. P. (1999) Science 285(5425), 245-248.
Palmieri, D., Watson, J. M., and Rinehart, C. A. (999) Exp Cell Res 247(1), 142-147.
Pignolo, R. J., Cristofalo, V. J. and Rotenberg, M. O. (1993) J. Biol. Chem. 268, 8949-8957.
Popovic, D., El-Shami, K. M., Vadai, E., Feldman, M., Tzehoval, E. and Eisenbach, L. (1998) Antimetastatic vaccination against Lewis lung carcinoma with autologous tumor cells modified to express murine interleukin 12. Clin. Exp. Metastasis 16, 623-632.
Steele, F. R., Chader, G. J., Johnson, L. V., and Tombran-Tink, J. (1993) Proc Natl Acad Sci USA 90(4), 1526-1530.
Stellmach, V. V., Crawford, S. E., Zhou, W., and Bouck, N. (2001) Proc Natl Acad Sci USA 98(5), 2593-2597.
Stratikos, E., Alberdi, E., Gettins, P. G., and Becerra, S. P. (1996) Protein Sci 5(12), 2575-2582.

1. Pigment epithelium derived factor (PEDF) isolated from plasma or a salt thereof having at least one PEDF biological activity selected from inhibitory activity of endothelial cell proliferation, anti-angiogenic activity, neurotrophic activity, neuronotrophic activity and gliastatic activity.

2. The PEDF according to claim 1, wherein the plasma is mammalian.

3. The PEDF according to claim 2, wherein the mammal is human.

4. An isolated fragment of PEDF according to claim 1 having at least one PEDF biological activity selected from inhibitory activity of endothelial cell proliferation, anti-angiogenic activity, neurotrophic activity, neuronotrophic activity and gliastatic activity.

5. The fragment according to claim 4, wherein t,he plasma is mammalian.

6. The fragment according to claim 5, wherein the mammal is human.

7. A derivative of PEDF according to claim 1 selected from phosphorylated, dephosphorylated, glycosylated, deglycosylated, cross-linked, methylated, sulfated, myristylated, hydroxylated, oxidized, reduced, acylated, ADP-ribosylated, amidated, cyclized and iodinated having at least one PEDF biological activity selected from inhibitory activity of endothelial cell proliferation, anti-angiogenic activity, neurotrophic activity, neuronotrophic activity and gliastatic activity.

8. The derivative according to claim 7, wherein the plasma is mammalian.

9. The derivative according to claim 8, wherein the mammal is human.

10. Isolated PEDF derived from liver cells, a fragment, a derivative, or a salt thereof having at least one PEDF biological activity selected from inhibitory activity of endothelial cell proliferation, anti-angiogenic activity, neurotrophic activity, neuronotrophic activity and gliastatic activity.

11. The PEDF according to claim 10, wherein the liver cells are from a human source.

12. A pharmaceutical composition comprising as an active ingredient PEDF isolated from plasma or a salt thereof according to claim 1 and a pharmaceutically acceptable carrier.

13. The pharmaceutical composition according to claim 12, wherein the plasma is mammalian.

14. The pharmaceutical composition according to claim 13, wherein the mammal is human.

15. A pharmaceutical composition comprising as an active ingredient an isolated fragment of PEDF from plasma or a salt thereof according to claim 4 and a pharmaceutically acceptable carrier.

16. The pharmaceutical composition according to claim 15, wherein the plasma is mammalian.

17. The pharmaceutical composition according to claim 16, wherein the mammal is human.

18. A pharmaceutical composition comprising as an active ingredient a derivative of PEDF from plasma or a salt thereof according to claim 7 and a pharmaceutically acceptable carrier.

19. The pharmaceutical composition according to claim 18, wherein the plasma is mammalian.

20. The pharmaceutical composition according to claim 19, wherein the mammal is human.

21. A pharmaceutical composition coinprising as an active ingredient the isolated PEDF derived from liver cells, a fragment, a derivative, or a salt thereof according to claim 10 and a pharmaceutically acceptable carrier.

22. The pharmaceutical composition according to claim 21, wherein the liver cells are from a human source.

23. A process for isolation of PEDF, which comprises subjecting plasma to protein precipitation and chromatography.

24. The process according to claim 23, wherein the plasma is mammalian.

25. The process according to claim 24, wherein the mammal is human.

26. A process according to claim 23 comprising the steps:
(i) subjecting plasma to protein precipitation, separating the protein precipitate, resuspending the precipitate, and subjecting the resuspended precipitate to ion exchange chromatography;
(ii) detecting the presence of PEDF in eluted fractions of step (i), collecting fractions containing PEDF activity, and subjecting the pooled fractions to dialysis;
(iii) subjecting the dialysate of step (ii) to affinity chromatography; and
(iv) repeating ion exchange chromatography and affinity chromatography as necessary to obtain homogeneous PEDF.

27. The process according to claim 26, wherein the plasma is mammalian.

28. The process according to claim 27, wherein the mammal is human.

29. A method for treating an angiogenesis-related disease in a patient comprising administering to the patient a therapeutic amount of a pharmaceutical composition according to claim 12.

30. The method according to claim 29, wherein the PEDF is isolated from mammalian plasma.

31. The method according to claim 30, wherein the mammalian plasma is human plasma.

32. The method according to claim 29, wherein the angiogenesis-related disease is selected from age-related macular degeneration, diabetic retinopathy, retinal detachment, retinal tumors, choroidal tumors, non-neuronal tumors, and nietastases.

33. A method for treating an angiogenesis-related disease in a patient comprising administering to the patient a therapeutic amount of a pharmaceutical composition according to claim 15.

34. The method according to claim 33, wherein the fragment of PEDF is isolated from mammalian plasma.

35. The method according to claim 34, wherein the mammalian plasma is human plasma.

36. The method according to claim 33, wherein the angiogenesis-related disease is selected from age-related macular degeneration, diabetic retinopathy, retinal detachment, retinal tumors, choroidal tumors, non-neuronal tumors, and metastases.

37. A method for treating an angiogenesis-related disease in a patient comprising administering to the patient a therapeutic amount of a pharmaceutical composition according to claim 18.

38. The method according to claim 37, wherein the PEDF is isolated from mammalian plasma.

39. The method according to claim 38, wherein the mammalian plasma is human plasma.

40. The method according to claim 39, wherein the angiogenesis-related disease is selected from age-related macular degeneration, diabetic retinopathy, retinal detachment, retinal tumors, choroidal tumors, non-neuronal tumors, and metastases.

41. A method for treating an angiogenesis-related disease in a patient comprising administering to the patient a therapeutic amount of a phannaceutical composition according to claim 21.

42. The method according to claim 41, wherein the liver cells are from a human source.

43. The method according to claim 41, wherein the angiogenesis-related disease is selected from age-related macular degeneration, diabetic retinopathy, retinal detachment, retinal tumors, choroidal tumors, non-neuronal tumors, and metastases.

44. A method for treating a neuronal disease in a patient comprising administering to the patient a therapeutic amount of a pharmaceutical composition according to claim 12.

45. A method for treating a neuronal disease in a patient comprising administering to the patient a therapeutic amount of a pharmaceutical composition according to claim 15.

46. A method for treating neuronal disease in a patient comprising administering to the patient a therapeutic amount of a pharmaceutical composition according to claim 18.

47. A method for treating neuronal disease in a patient comprising administering to the patient a therapeutic amount of a pharmaceutical composition according to claim 21.

48. A method for diagnosing the presence or incipience of angiogenesis-related disease in a subject comprising:
a. measuring the level of PEDF in a blood sample of said individual;
b. comparing it to the levels within a normal range of healthy control individuals; and
c. evaluating the PEDF levels in said samples, wherein a decrease in the PEDF level in the tested individual in comparison to the PEDF level in the said normal range indicates that said tested individual has a likelihood for developing an angiogenesis-related disorder.
